(19) United States
(12) Patent Application Publication
(10) Pub. No.: US 2010/0249815 A1
(43) Pub. Date: Sep. 30, 2010

(54) EVERTED SHEATH THROMBECTOMY DEVICE

(75) Inventors: Alexandra E. Jantzen, Bloomington, IN (US); Fred T. Parker, Unionville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/411,049

(22) Filed: Mar. 25, 2009

(51) Int. Cl. A61B 17/22

(52) U.S. Cl. 606/159

(57) ABSTRACT

A medical device for removing a thrombus includes an outer sheath and an inner catheter. The inner catheter is disposed within the inner lumen of the outer sheath. The device also includes a rollsock which is everted upon itself and connected to the distal ends of the outer sheath and inner catheter. A scraping device is attached to the outer surface of the rollsock, such that when the outer sheath is moved relative to the inner catheter the scraping device is exposed to an inner surface of a body vessel.

### BACKGROUND

The present invention relates generally to medical devices and more particularly to a device for breaking down and capturing a thrombus.

A thrombus is a deposit of clotted blood, which may be attached to a body vessel wall or occur elsewhere in the circulatory system. Because thrombi partially or fully obstruct blood flow through a body vessel, a method for removing thrombi is desired.

Traditionally, a thrombus is removed in a two-part procedure. First, the thrombus must be broken down. Typically, a thrombus is broken down by either a motor-turned wire which scrapes the inner wall of a body vessel or through the use of ultrasonic waves. Once the thrombus is broken down, it is removed by employing an aspirating device.

A thrombus may be broken down by a motor-turned wire as follows. A motor-turned wire is advanced to a treatment site by typical catheter procedures. The motor-turned wire is disposed within a sheath as it is advanced to the treatment site. Once the motor-turned wire reaches the treatment site, the motor-turned wire is removed from the sheath, exposing the motor-turned wire to the inner surface of a body vessel and the thrombus. In many embodiments of the motor-turned wire, the wire is outwardly-disposed such that, when the wire is moved beyond the distal portion of the sheath, the distal ends of the motor-turned wire are urged into contact with the body vessel wall. The motor is then turned, spinning the wire within the body vessel such that the wire contacts the inner surface of the body vessel and the thrombus. The wire may also be manually actuated. The spinning action of the wire breaks down the thrombus. Suction or an aspirating device may then be applied to the lumen within the sheath, urging the broken-down thrombus through the lumen and eventually outside the body vessel. As previously described, a thrombus may also be broken down through the use of ultrasonic waves.

The traditional methods of breaking down a thrombus using a motor-turned wire or ultrasonic waves require expensive capital equipment and maintenance. Thus, a device which breaks down a thrombus that does not include expensive capital equipment is desired. Additionally, a disposable device is desired to reduce time and money spent on maintenance.

### BRIEF SUMMARY

The invention may include any of the following aspects in various combinations and may also include any other aspect described below in the written description or in the attached drawings.

A medical device for performing a thrombectomy includes an outer sheath and an inner catheter, where the inner catheter is disposed within the inner lumen of the outer sheath. A flexible rollsock is connected to the distal portions of the outer sheath and inner catheter. The rollsock is everted upon itself. A scraping device is attached to the outer surface of the rollsock. The outer sheath may be moved relative to the inner catheter such that the scraping device is exposed to an inner surface of a body vessel. The inner catheter may also be moved relative to the outer sheath.

The scraping device may be a plurality of coils, a plurality of barbs, or any other suitable device.

The scraping device may be attached to the rollsock with adhesive, circumferential sutures, fibers, polyurethane coating, or any other suitable material.

The rollsock may be made of a polymer and the scraping device attached to the outer surface of the rollsock by heating the polymer and pressing the scraping device into the outer surface of the rollsock.

Suction may applied to the inner lumen of the outer sheath to urge a thrombus into the inner lumen of the outer sheath. Alternatively, suction may be provided through use of an external suction catheter disposed within the annular space between the outer sheath and the inner surface of a body vessel. The external catheter may be separate from the everted sheath thrombectomy device, or it may be attached to the outer surface of the outer sheath of the everted sheath thrombectomy device.

The annular space between the outer sheath and the inner catheter of the medical device for removing a thrombus may also be inflated such that the rollsock is forced outside said distal end of said outer sheath and urged into contact with the body vessel.

Alternatively, the rollsock may be composed of a plurality of separated sections. A scraping device is attached to the outer surface of the rollsock. An outwardly oriented actuator may be disposed within the annular space between the outer sheath and the inner catheter and within the everted portion of the rollsock such that when the outwardly oriented actuator is slidably positioned such that the distal portion of the outwardly oriented actuator is positioned outside the distal end of the outer sheath, the outwardly oriented actuator urges the outer portion of said rollsock toward the inner surface of a body vessel.

### BRIEF DESCRIPTION OF THE DRAWINGS

FIG. 1 is a side cross-sectional view of one embodiment of the everted sheath thrombectomy device wherein the scraping device is a plurality of barbs.

FIG. 2 is a side cross-sectional view of the everted sheath thrombectomy device wherein the annular space between the outer sheath and inner catheter is inflated to urge the scraping device toward the inner wall of a body vessel.

FIG. 3 is a side cross-sectional view of one embodiment of the everted sheath thrombectomy device wherein the rollsock is divided into a plurality of separated sections.

FIG. 4 is a cross-sectional view of the rollsock in one embodiment wherein the rollsock is divided into a plurality of separated sections.

FIG. 5A is a side cross-sectional view of one embodiment of the everted sheath thrombectomy device wherein the scraping device is a plurality of coils composed of at least one spring-shaped piece of wire.

FIG. 5B is a perspective view of one embodiment of the rollsock wherein the scraping device is a plurality of coils composed of at least one spring-shaped piece of wire.

FIG. 5C is a side cross-sectional view of one embodiment of the everted sheath thrombectomy device wherein the scraping device is a plurality of coils composed of several embolization coils.

FIG. 6 is a cross-sectional view of the rollsock in one embodiment wherein the scraping device is a plurality of coils.

FIG. 7A is a side cross-sectional view of the rollsock in one embodiment wherein the scraping device is a plurality of cut coils composed of at least one spring-shaped piece of wire or other material attached to the rollsock with sutures.

FIG. 7B is a side cross-sectional view of the rollsock in one embodiment wherein the scraping device is a plurality of cut coils composed of cut embolization coils attached to the rollsock with sutures.

FIG. 8 is a side cross-sectional view of the rollsock in one embodiment wherein the scraping device is a plurality of barbs having an eyelet for attaching the barbs to the rollsock with sutures.

FIG. 9 is a side cross-sectional view of one embodiment of the everted sheath thrombectomy device wherein an external catheter is disposed within the annular space between the outer catheter and inner surface of a body vessel to apply suction.

### DETAILED DESCRIPTION OF THE DRAWINGS AND THE PRESENTLY PREFERRED EMBODIMENTS

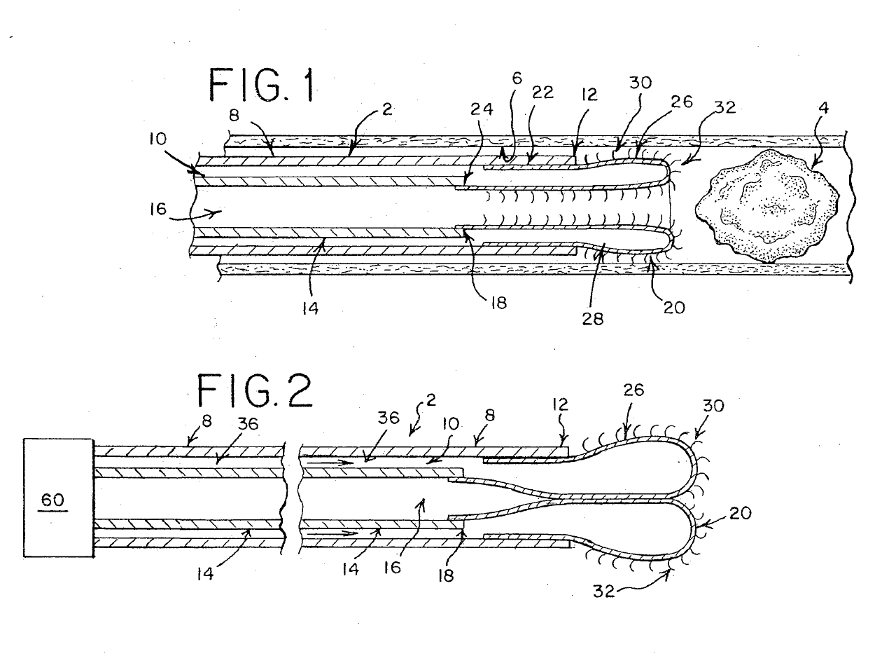

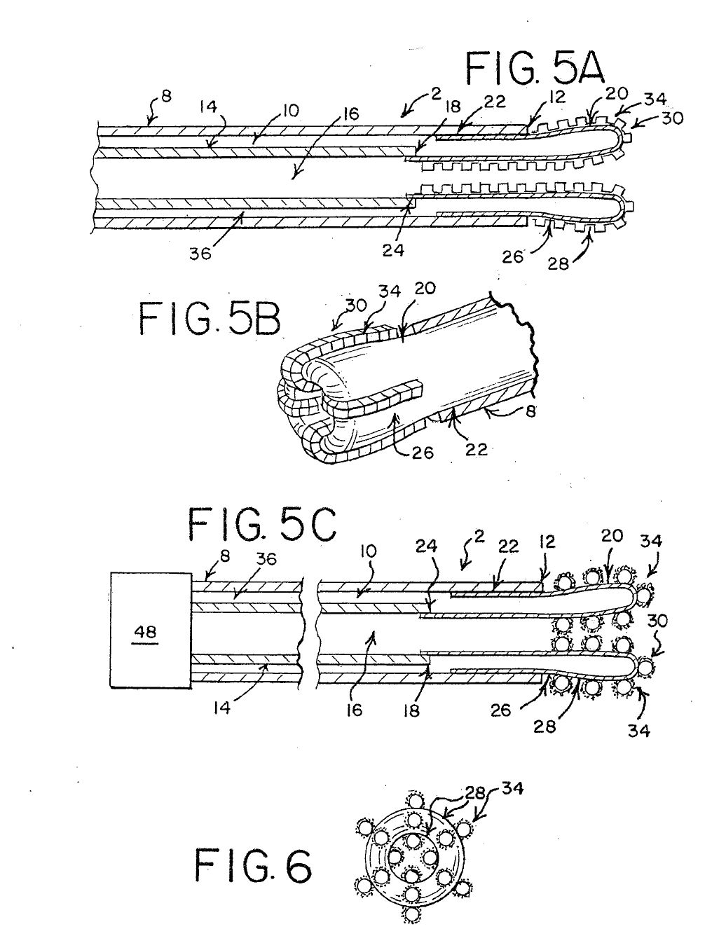

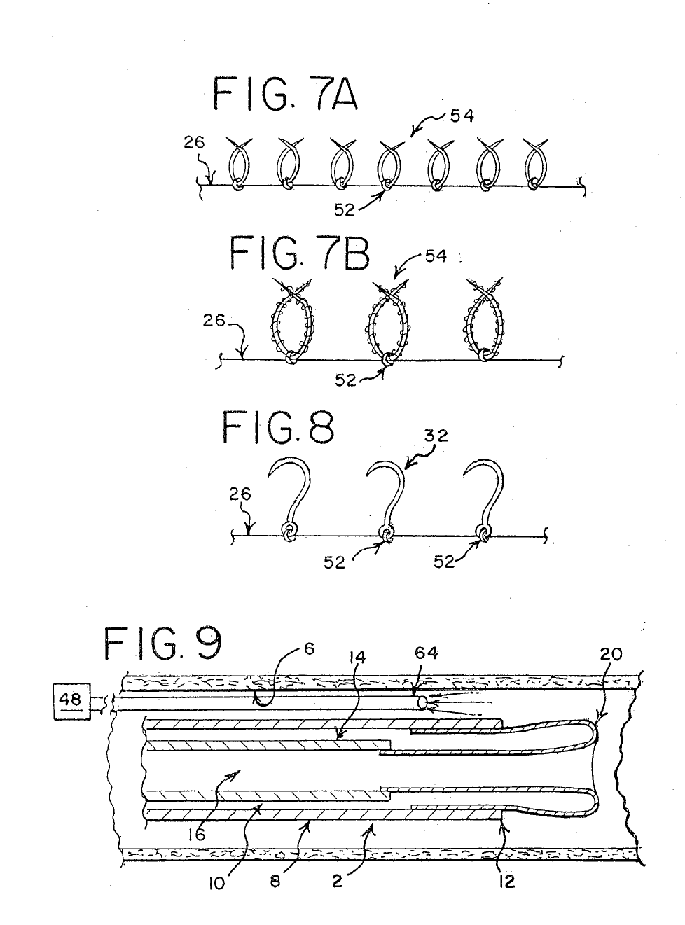

Referring now to the figures, an everted sheath thrombectomy device is shown. One embodiment of the device is shown in FIG. 1. An outer sheath 8 has an inner lumen 10 and a distal end 12. An inner catheter 14 is disposed within the inner lumen 10 of the outer sheath 8. The inner catheter 14 has an inner lumen 16 and a distal end 18. A rollsock 20, or tube of flexible material everted upon itself, is provided. The rollsock 20 may be made of Teflon, a polymer, or any other flexible material. The first end 22 of the rollsock 20 is attached to the distal end 12 of the outer sheath 8. The second end 24 of the rollsock 20 is attached to the distal end 18 of the inner catheter 14. The second end 24 of the rollsock 20 may be attached to any suitable surface of inner catheter 14, including surfaces on both the interior and exterior of inner catheter 14. The first end 22 of the rollsock 20 may also be attached to the distal end 12 of the outer sheath 8 such that the first end 22 of the rollsock 20 lines the inside of the outer sheath 8. The rollsock 20 has an outer surface 26. Because the rollsock 20 is everted upon itself, the portion of the outer surface 26 proximate to the first end 22 of the rollsock 20 faces the inner surface of a body vessel 6, and the portion of the outer surface 26 of the rollsock 20 proximate to the second end 24 of the rollsock 20 faces the interior of the everted sheath thrombectomy device 2. The doubled-over portion of the everted rollsock 20 defines an everted portion of the rollsock 28. A scraping device 30 is attached to the outer surface 26 of the rollsock 20. The scraping device 30 may be a plurality of barbs 32 as show in FIGS. 1-4, a plurality of coils 34 as shown in FIGS. 5-6, a plurality of cut coils 54 as shown in FIG. 7, or any other devices that may loosen a thrombus 4.

The plurality of barbs 32 may be straight, C-shaped, or any other shape effective to scrape and loosen a thrombus. The plurality of coils 34 may be composed of several different types of coils. The plurality of coils 34 may be composed of at least one spring-shaped piece of wire or other material, as shown in FIGS. 5A and 5B. The wire may be coiled in a circular fashion, or it may be repeatedly bent inward to form coils with straight sides. The wire may be a flat or round wire, and may be composed of steel or any other suitable metal. Additionally, as shown in FIG. 5C, the plurality of coils 34 may be composed of several individual embolization coils which are well-known in the field of medical devices. Additionally, the scraping device 30 may consist of a plurality of cut coils 54 created by attaching at least one spring-shaped piece of wire or other material or a plurality of embolization coils to the rollsock 20 and cutting through the top portion of the coils to create two barbs facing in opposite directions from each coil, as shown in FIGS. 7A and 7B. The stiffness of the scraping device 30 is selected based on the type of thrombus 4 to be removed. If the thrombus 4 is a chronic case and therefore more difficult to break down, removal of the thrombus 4 will likely require that the scraping device 30 be composed of a stiff material, for example stainless steel. If the thrombus 4 is a fresh or soft thrombus, the scraping device 30 may be made out a more flexible material, for example Dacron fibers or polymer loops. The scraping device 30 may be made of any material suitable for scraping and capturing a thrombus 4.

The scraping device 30 may be attached to the rollsock 20 using adhesive, polyurethane, or sutures, among other methods. If the scraping device 30 is composed of a plurality of barbs 32, an eyelet 62 may be included at one end of each of the plurality of barbs 32 such that sutures 52 may be passed through the eyelet 62 to secure each of the plurality of barbs 32 to the outer surface 26 of the rollsock 20, as shown in FIG. 8. If the scraping device 30 is composed of a plurality of coils 34, sutures 52 may be passed through the center of each of the plurality of coils 34 to secure each of the plurality of coils to the outer surface 26 of the rollsock 20. Sutures 52 may be composed of any suitable material, including suture material. Additionally, the rollsock 20 may be made of a polymer. If the rollsock 20 is made of a polymer, the scraping device 30 may be attached to the rollsock 20 by heating the rollsock 20 and pressing the scraping device 30 into the rollsock 20. The scraping device 30 may also be attached to the rollsock 20 by coating the outer surface 26 of the rollsock 20 and the scraping device 30 with a polyurethane coating.

The everted sheath thrombectomy device 2 is advanced to a treatment site using well-known stent introduction methods, including the use of a wire guide and/or a guide catheter. The wire guide is preferably removed before the thrombectomy procedure begins. Once the everted sheath thrombectomy device 2 reaches the treatment site, it is positioned adjacent to a thrombus 4. Alternatively, the everted sheath thrombectomy device 2 may be advanced past a thrombus 4 in the distal direction. The outer sheath 8 is then moved in the proximal direction relative to the inner catheter 14 such that the rollsock 20 rolls upon itself and exposes the scraping device 30 to the inner surface of a body vessel 6. Alternatively, the inner catheter 14 may be moved in the distal direction relative to the outer sheath 8. The scraping device 30 then engages the thrombus 4 and/or the inner surface of a body vessel 6. The scraping device 30 breaks down and/or captures the thrombus 4. Once the thrombus 4 is broken down or captured by the scraping device 30, the outer sheath 8 may be moved in the distal direction relative to the inner catheter 14 such that the rollsock 20 rolls back upon itself and the scraping device 30 urges the thrombus 4 into the inner lumen 10 of the outer sheath 8. Alternatively, the inner catheter 14 may be moved in the proximal direction relative to the outer sheath 8. Suction may also be applied to the inner lumen 16 of the inner catheter 14 to urge the thrombus 4 into the inner lumen 16 of the catheter 14 by placing a suction device 48 in communication with inner lumen 16. The suction device 48 can be any of a variety of suction devices that are well-known in the art, including aspirators. Suction may be applied by removing the wire guide. Alternatively, an external catheter disposed outside the everted sheath thrombectomy device 2 in the annular space between the outer sheath 8 and the inner surface of a body vessel 6 could be employed to apply suction. The external catheter could be separate from the everted sheath thrombectomy device 2 or attached to the outer surface of the outer sheath 8. Once the thrombus 4 is captured within the outer sheath 8, the everted sheath thrombectomy device 2 may be advanced to a new treatment site and used to break down and capture another thrombus.

As shown in FIG. 2, the annular space 36 between the outer sheath 8 and the inner catheter 14 may be inflated with a liquid such that the outer surface 26 of the rollsock 20 is urged toward the inner surface of a body vessel 6 by placing an inflating device 60 in communication with the annular space 36. Inflating device 60 may be any of a variety of devices that are well-known in the art, such as devices used for standard angioplasty procedures including a syringe and a syringe/crank assembly. It is preferable to choose a compliant material for the rollsock 20 when inflating the annular space 36, although any material otherwise suitable for the rollsock 20 may be used. The pressure to which rollsock 20 may be inflated is determined based on the material used to compose rollsock 20. The annular space 36 may be deflated after the scraping device 30 on the outer surface 26 of the rollsock 20 breaks down or captures a thrombus 4. The outer sheath 8 may then be moved relative to the inner catheter 14 such that the rollsock 20 is rolled back upon itself and the thrombus 4 is urged into the inner lumen 10 of the outer sheath 8. Alternatively, the inner catheter 14 may be moved relative to the outer sheath 8. Suction may also be applied to the inner lumen 10 of the outer sheath 8 to urge the thrombus 4 into the inner lumen 10 of the outer sheath 8 using any of the previously described methods of applying suction. Once the thrombus 4 is captured within the outer sheath 8, the everted sheath thrombectomy device 2 may be advanced to a new treatment site and used to break down and capture another thrombus.

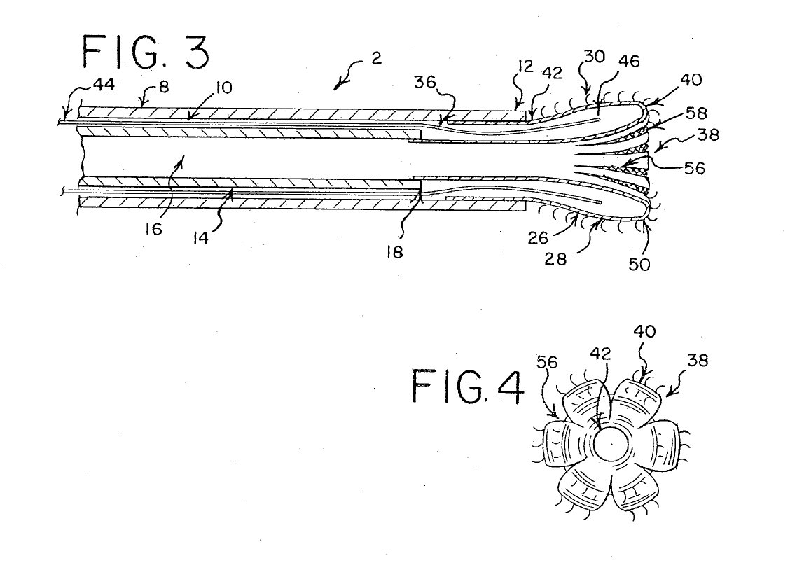

An alternative embodiment of the everted sheath thrombectomy device 2 is shown in FIGS. 3 and 4. As shown in FIG. 4, a rollsock with separated sections 50 is divided into a plurality of separated sections 38. The plurality of separated sections 38 is created by cutting slits 56 in the rollsock to define a series of separated sections 50. The slits 56 remain open to facilitate the rolling action of the rollsock with separated sections 50. Alternatively, a webbing section 58 may be disposed between each of the plurality of separated sections 38 to seal the slits 56 between the separated sections 50. Webbing section 58 is preferably composed of a highly compliant material. An outwardly oriented actuator 44 is disposed within the annular space 36 between the outer sheath 8 and the inner catheter 14, such that when the distal portion 46 of the outwardly oriented actuator 44 is slidably positioned in the distal direction and the distal portion 46 of the outwardly oriented actuator 44 is positioned outside the distal portion 12 of the outer sheath 8, the outwardly oriented actuator 44 urges the outer surface 26 of the rollsock with separated sections 50 toward the inner surface of a body vessel 6. The scraping device 30 may then engage a thrombus 4 to break down and/or capture the thrombus 4. The outer sheath 8 is then moved in the distal direction relative to the inner catheter 14 such that the rollsock with separated sections 50 is rolled back upon itself and the thrombus 4 is urged into the inner lumen 10 of the outer sheath 8. Alternatively, the inner catheter 14 may be moved in the proximal direction relative to the outer sheath 8. Suction may also be applied to the inner lumen 10 of the outer sheath 8 by suction device 48 to urge a thrombus 4 into the inner lumen 10 of the outer sheath 8 or in any other method previously described. Once the thrombus 4 is captured within the outer sheath 8, the everted sheath thrombectomy device 2 may be advanced to a new treatment site and used to break down and capture another thrombus.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

We claim:

1. A medical device comprising:
an outer sheath having a distal end and an inner lumen;
an inner catheter having a distal end and an inner lumen, wherein said inner catheter is disposed within said inner lumen of said outer sheath;
a rollsock with a first end and a second end and an outer surface;
said distal end of said outer sheath connected to said first end of said rollsock;
said distal end of said inner catheter connected to said second end of said rollsock;
said rollsock everted upon itself;
a scraping device attached to said outer surface of said rollsock;
wherein said outer sheath may be moved relative to said inner catheter such that said scraping device is exposed to an inner surface of a body vessel.

2. The medical device of claim 1 wherein said scraping device is selected from the group consisting of a plurality of coils, a plurality of barbs, and a plurality of cut coils.

3. The medical device of claim 1 wherein said rollsock is composed of Teflon.

4. The medical device of claim 1 wherein said scraping device is attached to said rollsock with adhesive.

5. The medical device of claim 1 wherein said scraping device is attached to said rollsock with sutures.

6. The medical device of claim 1 wherein said scraping device is attached to said outer surface of said rollsock by covering said outer surface of said rollsock and said scraping device with a polyurethane coating.

7. The medical device of claim 1 wherein said rollsock is a polymer, and said scraping device is attached to said outer surface of said rollsock by heating said polymer and pressing said scraping device into said outer surface of said rollsock.

8. The medical device of claim 1 further comprising a suction device in communication with said inner lumen of said inner catheter.

9. The medical device of claim 1 further comprising an inflating device in communication with an annular space between said outer sheath and said inner catheter such that said inflating device inflates said annular space.

10. The medical device of claim 1 further comprising a suction device in communication with an external catheter, said external catheter disposed in an annular space between said inner surface of a body vessel and said outer sheath.

11. The medical device of claim 1 wherein said rollsock is composed of Teflon, said scraping device is selected from a group consisting of a plurality of coils, a plurality of barbs, and a plurality of cut coils, and said scraping device is attached to said rollsock with sutures.

12. A medical device comprising:
an outer sheath having a distal end and an inner lumen;
an inner catheter having a distal end and an inner lumen, wherein said inner catheter is disposed within said inner lumen of said outer sheath;
a rollsock with a first end and a second end, and an outer surface and an everted portion;
said distal end of said outer sheath connected to said first end of said rollsock;
said distal end of said inner catheter connected to said second end of said rollsock;
said rollsock everted upon itself;
a scraping device attached to said outer surface of said rollsock;
wherein said outer sheath may be moved relative to said inner catheter such that said scraping device is exposed to an inner surface of a body vessel;
said rollsock composed of a plurality of separated sections;
said separated sections at least partially disposed within said everted portion of said outer surface;
and an outwardly oriented actuator disposed within an annular space between said outer sheath and said inner catheter and within said everted portion of said rollsock, said outwardly oriented actuator having a distal portion, such that when said outwardly oriented actuator is slidably positioned such that said distal portion of said outwardly oriented actuator is disposed outside said distal end of said outer sheath, said outwardly oriented actuator urges said outer portion of said rollsock toward said inner surface of a body vessel.

12. The medical device of claim 12 wherein said scraping device is selected from the group consisting of a plurality of coils, a plurality of barbs, and a plurality of cut coils.

13. The medical device of claim 12 wherein said rollsock is composed of Teflon.

14. The medical device of claim 12 wherein said scraping device is attached to said rollsock with adhesive.

15. The medical device of claim 12 wherein said scraping device is attached to said rollsock with sutures.

16. The medical device of claim 12 further comprising a suction device in communication with said inner lumen of said inner catheter.

17. The medical device of claim 12 further comprising an inflating device in communication with an annular space between said outer sheath and said inner catheter such that said inflating device inflates said annular space.

18. The medical device of claim 12 further comprising a suction device in communication with said inner lumen of said inner catheter, wherein said rollsock is composed of Teflon and said scraping device is selected from a group consisting of a plurality of coils, a plurality of barbs, and a plurality of cut coils.

19. The medical device of claim 12 further comprising at least one webbing section disposed between each of said plurality of separated section such that said everted portion of said rollsock is sealed.
